(19) United States
(12) Patent Application Publication
(10) Pub. No.: US 2025/0360231 A1
(43) Pub. Date: Nov. 27, 2025

(54) MARKER FOR USE IN IDENTIFYING OR PREDICTING ANGIOGENESIS

(71) Applicant: NOVAI LTD, Reading (GB)

(72) Inventors: Maria Francesca Cordeiro, Bray Berkshire (GB); John Madison, Crowborough Sussex (GB)

(21) Appl. No.: 18/255,607

(22) PCT Filed: Dec. 3, 2021
(86) PCT No.: PCT/EP2021/084119

§ 371 Date: Jun. 2, 2023

(30) Foreign Application Priority Data
Dec. 3, 2020 (GB) 2019109.4

(51) Int. Cl. A61K 49/00; C07K 14/47

(52) U.S. Cl. CPC A61K 49/0056; A61K 49/0021; C07K 14/4721

(57) ABSTRACT

The present invention provides a labelled cell stress marker for use in identifying or predicting angiogenesis in a subject. Also provided is a method of identifying or predicting angiogenesis in a subject, the subject having been administered a labelled cell stress marker. The method comprises the steps of (a) generating an image from the subject using a first imaging device and (b) identifying labelled cell stress marker positive cell(s) in the image. Angiogenesis is identified or predicted if labelled cell stress marker positive cell(s) are identified in the image.

### TECHNICAL FIELD

This invention relates to a labelled cell stress marker for use in identifying or predicting angiogenesis in a subject. The invention also relates to methods of identifying or predicting angiogenesis in a subject.

### BACKGROUND

Angiogenesis is a key pathology associated with conditions such as cancer, allergic disease, autoimmune disease and ocular conditions such as age-related macular degeneration (AMD), in particular wet-AMD. Wet AMD is a major cause of vision loss, affecting 2.5% of those aged 65 or over in the UK. This rises to 6.3% in those aged 80 and above in the UK.

Current diagnosis of wet-AMD relies upon imaging the retina to assess for the development of abnormal blood vessels. However, wet-AMD has a silent and rapidly progressive nature, often resulting in diagnosis and treatment being carried out too late to be effective. This can lead to significant morbidity.

There remains a need to identify subjects at risk of developing wet-AMD, so that treatment can be administered early. The present invention seeks to address these and other issues.

### SUMMARY OF THE INVENTION

According to a first aspect of the invention, there is provided a labelled cell stress marker for use in identifying or predicting angiogenesis in a subject.

As used herein, the term “cell stress marker” refers to a marker that identifies cells undergoing cell stress. Thus, the marker may be known as a compound or molecule that specifically binds to stressed cells. The cell stress may be predictive of the cell ultimately undergoing apoptosis. In some embodiments, the cell stress marker is also capable of labelling apoptosing cells (i.e. the marker is capable of labelling cells undergoing cell stress, as well as apoptosing cells). Thus, in embodiments, the cell stress marker may be referred to herein as an “apoptosis marker”.

As used herein, the term “apoptosis marker” refers to a marker that allows cells undergoing apoptosis to be distinguished from live cells. The marker may also be able to distinguish apoptosing cells from necrotic cells. Thus, the marker may be known as a compound or molecule that specifically binds to apoptotic cells.

The cell stress marker may comprise or consist of a marker that identifies stressed cell membranes.

The term “angiogenesis” is a known term of the art which refers to the growth of new blood vessels from existing vasculature. Advantageously, the present inventors have found that labelled cell stress markers can be used to identify stressed cells at a single cell level to predict or identify angiogenesis. In some embodiments, the angiogenesis is early-stage angiogenesis. By identifying angiogenesis at an early stage, or predicting the development of angiogenesis, the inhibition or promotion of angiogenesis can be clinically targeted at an early stage to ensure maximal efficiency for the subject.

By “predicting angiogenesis”, this may be understood to refer to a prediction of the development of angiogenesis, for example before angiogenesis has occurred. This allows clinical intervention at an unexpectedly early stage. This, in turn, prevents the rapid progression of symptoms, such as, for example, vision loss in wet-AMD.

It is entirely unexpected that the cell stress marker can identify or predict angiogenesis. This is because angiogenesis relates to new cell growth, rather than cell stress and/or apoptosis.

In some embodiments, the cell stress marker is an annexin or a fragment or variant thereof. The annexin family of proteins are known apoptotic markers. Annexins are proteins that bind reversibly to cellular membranes in the presence of cations. Annexins useful in the invention may be natural or may be recombinant.

By “fragment” this will be understood to refer to a truncated version of an annexin that substantially retains the functional activity of the whole annexin. By “substantially retains”, this will be understood to refer to a functional activity of at least 80%, at least 85%, at least 90%, at least 95%, at least 97%, at least 98% or at least 99% of the whole annexin. A variant of an annexin will be understood to refer to a protein which has an amino acid sequence that varies from the wild type annexin, such that the variant has at least 80%, at least 85%, at least 90%, at least 95%, at least 97%, at least 98% or at least 99% sequence identity to the wild type annexin. A variant of an annexin will substantially retain the functional activity of the wild type annexin.

The annexin may be annexin 5, 11, 2 or 6. In some embodiments, the annexin is annexin 5. In another embodiment, the annexin is annexin 128 (Tait et al. 2005). Annexin 128 is a variant of annexin 5 and differs from the wild-type annexin 5 by two single amino acid mutations. Annexin 128 includes an exposed thiol group at the N-terminus, which affords increased conjugation efficiency for molecular tags, such as fluorescent labels.

Without being bound by theory, the present inventors believe that phosphatidylserine, to which annexin is capable of binding, is exposed in stressed endothelial cells. Therefore, the identification of phosphatidylserine, for example by the binding of annexin, can indicate cell stress. The inventors have surprisingly found that the exposure of phosphatidylserine on the cell membrane can be indicative of the later development of early stage angiogenesis.

Other apoptosis markers are known in the art including, for example C2A domain of synaptotagmin-I, duramycin, non-peptide based isatin sulfonamide analogs, such as WC-11-89, and ApoSense, such as NST-732, DDC and ML-10 (Saint Hubert et al., 2009). Such apoptotic markers are suitable for use as cell stress markers of the present invention.

The label of the cell stress marker may be visible. Exemplary visible labels may include, but not necessarily be limited to quantum dots, nanospheres and/or nanorods. Other visible labels will be known to the skilled person.

The label may be fluorescent. Fluorescent labels refer to compounds or molecules (such as fluorophores) which emit light in response to excitation, and which may be selected for use due to increased signal-to noise ratio and thereby improved image resolution and sensitivity while adhering to light exposure safety standards to avoid phototoxic effects. It is preferred that the fluorescent label causes little or no inflammation on administration. The fluorescent label may have a wavelength in the infra-red or near infra-red spectrum. The fluorescent label may have an emission wavelength of about 400 nm to about 1000 nm, preferably about 500 nm to about 900 nm, more preferably about 700 nm to about 900 nm.

Suitable fluorescent labels include one or more of sodium fluorescein, indocyanine green (ICG), curcumin, IRDye700, Dy-776 (which may otherwise be referred to as D-776), Dy-488 and Dy-781. In some embodiments the fluorescent label is D-776.

The labelled cell stress marker may be prepared using standard techniques for conjugating a label, for example a fluorescent label, to a marker compound. Such labels may be obtained from well-known sources such as Dyomics. Appropriate techniques for conjugating the label to the marker are known in the art and may be provided by the manufacturer of the label.

It will be appreciated that angiogenesis (optionally early-stage angiogenesis) can be associated with pathological conditions, such as diseases, as well as normal growth and development. Thus, the angiogenesis may be physiological, therapeutic or pathological angiogenesis. In the context of the present invention, physiological angiogenesis will be understood to refer to angiogenesis associated with normal growth and healing in a subject. For example, angiogenesis has previously been shown to be induced by exercise, particularly endurance exercise at high altitude, e.g. mountain climbing. Thus, the physiological angiogenesis may be associated with previous exercise by the subject. The therapeutic angiogenesis may be associated with wound healing and/or integration of a tissue engineered scaffold.

In some embodiments, the angiogenesis is pathological angiogenesis. By pathological angiogenesis, this will be understood to be angiogenesis which is associated with or implicated in a disease. The pathological angiogenesis may be associated with a disease, a stage of disease, a disease severity and/or predictive of disease developing.

The cell stress marker may be for use in identifying or predicting pathological angiogenesis. The identification or prediction of pathological angiogenesis by the cell stress marker may predict or identify a particular disease, stage of disease and/or disease severity. Advantageously, the ability to predict a particular disease, stage of disease and/or disease severity, or to identify a particular disease at an early stage, allows the most suitable treatment course to be selected, administered and, optionally, monitored.

In the context of disease, the term “predict” as used herein, may be understood to refer to a prediction of a disease, stage of disease and/or disease severity developing, before it has occurred, or at such an early stage of disease that the subject is asymptomatic and/or the disease would not otherwise be identified. In embodiments where early-stage angiogenesis is identified, this may be used to identify a disease at an early stage. For example, early-stage angiogenesis may be identified when the subject has no other symptoms of a disease, or when the subject has only limited or minor symptoms of a disease, such that they are at an early stage of the disease which may be otherwise difficult or not possible to diagnose. This is useful in enabling a clinician to select and administer a treatment at an early stage, thereby potentially avoiding unnecessary progression of the disease.

The disease may comprise any disease associated with angiogenesis. For example, the disease may comprise any disease for which anti-VEGF can be administered as a treatment. In some embodiments, the disease comprises cancer, autoimmune disease, cardiovascular disease, allergic disease, sickle cell disease, and/or ocular disease. It will be appreciated that some diseases may be, for example, an ocular disease and an autoimmune disease (such as diabetic retinopathy).

In some embodiments, angiogenesis is associated with cardiovascular disease. In other embodiments, angiogenesis is associated with effective treatment of a cardiovascular disease (i.e. therapeutic angiogenesis). An example of cardiovascular disease associated with angiogenesis comprises vein occlusion, for example, a stroke.

Various autoimmune diseases are well-known to those skilled in the art. They include, but are not limited to sarcoidosis, rheumatoid arthritis, multiple sclerosis (MS), psoriasis, diabetes (particularly type I diabetes), ulcerative colitis, systemic lupus erythematosus (lupus), Guillain-Barre syndrome, chronic inflammatory demyelinating polyneuropathy, Graves' disease, Hashimoto's thyroiditis, myasthenia gravis, autoimmune encephalitis, neuromyelitis optica and anti-myelin oligodendrocyte glycoprotein antibody disease (MOG).

In some embodiments, the autoimmune disease is a central nervous system (CNS) autoimmune disease. Exemplary CNS autoimmune diseases include, but are not limited to neurosarcoidosis, MS, neuromyelitis optica and anti-myelin oligodendrocyte glycoprotein antibody disease (MOG).

The allergic disease may comprise or consist of rhinitis, asthma and/or hayfever. Typically, the allergic disease is a chronic allergic disease. It has been found that chronic allergic disease can be associated with angiogenesis. By “chronic allergic disease”, this will be understood to refer to an allergic disease which is long-term and recurring. Long-term may refer to a condition persisting and/or recurring for at least 6 months, at least 1 year, at least 2 years, at least 5 years, at least 10 years or at least 20 years.

In some embodiments the disease comprises ocular disease. Ocular diseases include, but are not necessarily limited to retinal vein occlusion, diabetic retinopathy, age-related macular degeneration (AMD), optic neuritis and ocular vascular disease. In some embodiments, the ocular disease comprises ocular neurodegenerative disease. The term “ocular neurodegenerative disease” is well known to those skilled in the art and refers to disease caused by gradual and progressive loss of ocular neurons. They include, but are not limited to AMD, optic neuritis and diabetic retinopathy. Neurodegenerative diseases include, for example, Parkinson's disease, Alzheimer's disease, Huntington's disease and Friedreich's ataxia.

In some embodiments, the ocular disease comprises or consists of diabetic retinopathy or AMD.

In some embodiments the ocular disease comprises or consists of AMD. Optionally, the ocular disease comprises or consists of wet-AMD. The present inventors have surprisingly found that a labelled cell stress marker can be used to identify or predict angiogenesis in a subject, the identification or prediction of angiogenesis being a predictive marker of wet-AMD. This allows clinicians to predict if a subject will go on to develop wet-AMD. Wet-AMD symptoms can develop very quickly, potentially leading to a permanent loss of vision. Such a prediction is therefore particularly beneficial in enabling clinicians to administer treatment to a subject at an early stage, thereby reducing and in some embodiments avoiding the loss of vision.

In some embodiments, the subject is a mammal. For example, the subject may be a rat, mouse, human, rabbit, horse, dog, cat, cow or sheep. In some embodiments, the subject is selected from a mouse, rat, human and rabbit. In some embodiments, the subject is human.

According to a second aspect of the invention, there is provided use of a labelled cell stress marker for identifying or predicting angiogenesis in a subject. The angiogenesis may be physiological or therapeutic angiogenesis, preferably physiological angiogenesis, such as that described herein.

According to a third aspect of the invention, there is provided a method of identifying or predicting angiogenesis in a subject, the subject having been administered a labelled cell stress marker, comprising the steps of:
(a) generating a first image from the subject using a first imaging device;
(b) identifying labelled cell stress marker positive cell(s) in the image;
wherein angiogenesis is identified or predicted if labelled cell stress marker positive cell(s) are identified in the image.

In a further aspect of the invention, there is provided a method of identifying or predicting angiogenesis in a subject, comprising the steps of:
(a) administering a labelled cell stress marker to a subject;
(b) generating an image from the subject using an imaging device;
(c) identifying labelled cell stress marker positive cell(s) in the image;
wherein angiogenesis is identified or predicted if labelled cell stress marker positive cell(s) are identified in the image.

The present inventors have found that a labelled cell stress marker can be used to label cell stress at the single-cell level. The identification of cell stress at the single cell level advantageously predicts the subsequent development of angiogenesis and/or enables the detection of angiogenesis at an early stage, thereby enabling the prediction or identification of a disease before other or further symptoms have developed.

Administration of the labelled cell stress marker may be intravenous, intranasal, topical or intravitreal. By topical, this will be understood to refer to application to the surface of the subject, for example, to the skin or to the surface of the eye. In some embodiments, the labelled cell stress marker is administered intravenously. In other embodiments, the labelled cell stress marker is administered intravitreally.

In some embodiments, the method further comprises administering an appropriate treatment for the disease, stage of disease, severity of disease or predicted disease identified based on the angiogenesis or predicted angiogenesis. A suitable treatment may comprise or consist of an anti-angiogenic (i.e. a treatment that inhibits angiogenesis), for example an anti-VEGF compound. Exemplary anti-angiogenics may include, but not necessarily be limited to axitinib, bevacizumab, ranibizumab, cabozantinib, everolimus, lenalidomide, pazopanib, ramucirumab, regorafenib, sorafenib, sunitinib, thalomide, vandetanib and zib-aflibercept. Administration of the appropriate treatment may be intravenous, intranasal, topical or intravitreal.

In some embodiments, identifying labelled cell stress marker positive cell(s) comprises counting the number of labelled cell stress marker positive cell(s) in the image. Advantageously, this step does not need to be repeated. In other words, the step of identifying labelled cell stress marker positive cells need only occur once, since the inventors have surprisingly found that only one identification step is required to identify angiogenesis or predict its subsequent occurrence. Thus, the present methods are particularly quick, accurate and cost-effective.

In some embodiments, identifying labelled cell stress marker positive cell(s) comprises determining the location of labelled cell stress marker positive cell(s) in the subject. This may comprise determining the anatomical location of labelled cell stress marker positive cell(s) shown in the image. As mentioned previously, the cell stress marker of the invention may label both cells undergoing stress and apoptosing cells. Accordingly, it may be desirable to distinguish between apoptosing and stressed cells. The inventors have identified that one way of achieving this is to look at the location of the marked cell and consider whether it is in in a location where angiogenesis is likely to be taking place, for example near to a blood vessel, or if it is somewhere angiogenesis is unlikely to occur, e.g. in the photoreceptor layer of the retina. The methods of the invention may therefore further comprise the step of determining the location of the cell stress marker positive cell(s), the location being indicative of whether the cell is involved in angiogenesis or is apoptotic. This enables practitioners to reduce false positives from apoptosis-positive cells, thereby ensuring the identification and/or prediction of angiogenesis is especially accurate. The step of determining the location of the cell stress marker positive cell may comprise identifying the location of the cell relative to an anatomical structure, such as a blood vessel, or tissue type.

The step of determining the location may comprise identifying the location of the cell on the first image generated. Alternatively, it may comprise locating the cell in a second image, particularly an image showing the cell from a different aspect, or showing tissues or other structures that may not be visible in the first image. Optionally, the method comprises determining the location of labelled cell stress marker positive cell(s) in a plurality of planes. For example, the location of labelled cell stress marker positive cell(s) may be determined across an x axis and a y axis, e.g. in a 2D image. The location of labelled cell stress marker positive cell(s) may be determined across an x axis, a y axis and a z axis. In some embodiments, the location of the cell may be identified from a plurality of images. For example, the location of the cell may be identified using at least a second and third image. Each of the plurality of images may be taken using a similar or identical frame of reference, so that after the plurality of images are obtained, they can be stacked together to form a x, y and z axis. In other words, to form a 3D image. The 3D image can be used to identify the location of the cell.

The method may comprise generating the second image of the subject, for example using the same or a different imaging device, and, optionally comparing that image with the first image. Where the method comprises comparing one or more images, it is preferable that one image may be overlaid on the other. The method may comprise the step of doing so. It may also comprise the step of image registration, i.e. aligning the images, to correctly match up the cell stress marker positive cells shown in each image.

Where the method comprises the use of two or more images, the images may be obtained using the same or different imaging devices. For example, a first imaging device may comprise or consist of a confocal scanner laser microscope. An example of a confocal scanner laser microscope is a confocal scanning laser ophthalmoscope (cSLO).

In some embodiments a first and a second imaging device are used to obtain a plurality of images. The second imaging device may comprise or consist of a computerized tomography (CT) scanner, a magnetic resonance imaging (MRI) scanner, a position emission tomography (PET) scanner, a polariser, a reflective image scanner, fundus camera and/or an OCT scanner. Preferably, the second imaging device is a different device to the first imaging device.

In some embodiments, the second imaging device comprises or consists of a computerized tomography (CT) scanner, a magnetic resonance imaging (MRI) scanner, a position emission tomography (PET) scanner, and/or an OCT scanner.

In some embodiments, the second imaging device comprises or consists of an OCT scanner.

In some embodiments, a first, second and third imaging device are used to obtain a plurality of images. The third imaging device may comprise or consist of a computerized tomography (CT) scanner, a magnetic resonance imaging (MRI) scanner, a position emission tomography (PET) scanner, polariser, reflective image scanner, fundus camera and/or an OCT scanner. Preferably, the third imaging device is a different device to the first and second imaging device.

A first imaging device may be used to obtain a 2D or 3D image. Optionally, the first imaging device is used to obtain a 2D image. A second imaging device may be used to obtain a 2D or 3D image. The second imaging device may be used to obtain a 3D image. In some embodiments, a second or third imaging device is used to provide a mask image. By mask image, this will be understood to refer to a “background” image, i.e. an image that can be used to set a background value for the image which is used to identify the location of the cell.

In some embodiments, the third imaging device comprises or consists of a reflective image scanner.

In one embodiment, the method comprises determining the location of one or more blood vessel(s). Determination of the location of the blood vessels may be by angiography, methods for which are known in the art. For example, the location of the blood vessels may be determined using Fluorescein angiography (FA), indocyanine green angiography (ICGA), and/or optical coherence tomography (OCT), optionally optical coherence tomography angiography (OCTA).

In some embodiments, determining the location of labelled cell stress marker positive cell(s) in the image may comprise determining the location of said cells relative to the location of blood vessel(s). For example, when the labelled cell stress marker positive cell(s) are identified as being adjacent to or on a blood vessel, this may be used to identify cell stress rather than apoptosis. This, in turn, may be used as a positive indicator of early-stage angiogenesis or the predicted subsequent development of angiogenesis.

For example, in embodiments where the identification or prediction of angiogenesis is used to predict if a subject may develop an ocular neurodegenerative disorder, the determination of the location of the labelled cell stress marker positive cell(s) (optionally by further comprising determining the location relative to the location of blood vessel(s)), may be used to predict if the subject may develop wet-AMD as opposed to glaucoma. This is because wet-AMD is typified by cell stress, whereas glaucoma is associated with apoptosis. This is particularly beneficial since wet-AMD and glaucoma are both serious and rapidly progressing eye conditions that require different treatments. The ability to distinguish between the two conditions prior to the development of symptoms, or at an early stage of disease, ensures that the correct treatment can be chosen and administered to avoid unnecessary loss of vision.

The identification of labelled cell stress marker positive cell(s) and optionally the location of labelled cell stress marker positive cell(s) in the image may be computer-implemented. This may comprise generating training data for use in training an identification model, such as an artificial neural network. The artificial neural network may comprise a convolutional neural network (CNN). The training data may comprise control data manually classified as comprising or not comprising labelled cell stress marker positive cell(s), for example, from subjects already known to have (or not have) a disease, for example wet-AMD.

In some embodiments, an identification model may be trained using training data. This may be in addition to or instead of a step of generating training data for use in training an identification model. Once trained, the identification model may be validated manually.

A trained identification model (such as, for example, a convolutional neural network), may be used to identify labelled cell stress marker positive cell(s) and optionally the location of labelled cell stress marker positive cell(s).

Alternatively, the identification of labelled cell stress marker positive cell(s), and optionally the location of labelled cell stress marker positive cell(s) in the image may be manual.

In some embodiments, the identification of at least 5, at least 10, at least 15, at least 20, at least 30 or at least 40 labelled cell stress marker positive cells in the image is predictive of the subject developing a disease. In some embodiments, the identification of more than 5, more than 10, more than 15, more than 20, more than 30 or more 40 labelled cell stress marker positive cells in the image is predictive of the subject developing a disease. For example, the identification of at least 5, at least 10, at least 15, at least 20, at least 30, or at least 40 labelled cell stress marker positive cells in an image of an eye of the subject may be predictive of the subject developing wet-AMD. In some embodiments, the identification of at least 5, optionally more than 5, labelled cell stress marker positive cells in an image in an eye of the subject is predictive of the subject developing wet-AMD.

Advantageously, the present inventors have also found that an increasing number of labelled cell stress marker positive cells identified correlates with the severity of disease that subsequently develops, i.e. the higher the number of identified labelled cell stress marker positive cells in an image, the greater the severity of disease which will subsequently occur. Thus, in some embodiments, the identification of at least 10, at least 15, at least 20, at least 30 or at least 40 labelled cell stress marker positive cells in the image is predictive of the subject developing an increased severity of disease. This can advantageously be used to determine if the subject requires a higher dose of treatment, more regular administration of treatment or indeed a different treatment entirely.

In some embodiments, the step of generating an image is repeated to obtain a second, third, fourth, fifth or more additional images. An image may be compared to one or more preceding images of the same area of the subject, for example of the same eye of the subject. Repetition of generating the image may be every month, every two months, every six months, annually or biennial (once every two years). The method may comprise overlaying an image over one, two, three, four or more preceding images. Comparing images of the same area of the subject over time may be used to monitor the progression of disease. Alternatively, in embodiments where an appropriate treatment has been administered, comparing images of the same area of the subject over time may be used to monitor the efficacy of treatment administered. For example, after generating a first image, the subject may be administered an appropriate treatment. Any subsequent images obtained can be used to monitor the efficacy of said treatment.

In some embodiments, the method may be carried out on a subject on which another method has already been carried out to try to diagnose or predict the likelihood of the subject developing a particular disease. The present method can therefore be used as a “second opinion” to test and/or improve the accuracy of the another (first) method, and to ensure that any false negatives from the first method are identified.

According to another aspect of the invention, there is provided a labelled apoptosis marker for use in identifying apoptosis in a body part of a subject which is not an eye. In an embodiment, the body part is not part of the central nervous system. The body part may be all or part of a limb (for example an arm or a leg), a liver, a spleen, a heart, one or more lungs, a stomach, small intestine, large intestine, kidney, skin or bladder.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows that the DARC CNN system predicts new Subretinal Fluid (wet AMD) in OCT. Alignment application: for each eye, the reflective OCT localisation map (A) is aligned to the baseline autofluorescent image captured with confocal Scanning Laser Ophthalmoscope (cSLO) (B) and the OCT scan (C), utilising a bespoke application involving automatic landmarks defining OCT areas in serial scans ((filled white circles) and the manual placement of fiducial markers (yellow and blue crosses, A, B). The DARC image (D) is taken 240 minutes after intravenous Anx776 is administered with the cSLO, and is automatically aligned to the baseline autofluorescent image (B) which is now also aligned with the OCT images (A, C). Corresponding points in the DARC image can then be located in the OCT scan (C) using the cyan vertical line, allowing identification of all cross-sectional structures in the OCT image at that vertical location. Hence, in this example, at baseline, the location of the cyan encircled yellow DARC spot (D), can be recorded by x,y coordinates in all the retinal layers of the baseline OCT scan (C). This can be done for all DARC spots, with corresponding points located in all follow-up OCT scans. DARC spot localisation: DARC spots are automatically detected using a previously described CNN-aided algorithm (Lindekleiv et al. (2013)). The cyan encircled yellow DARC spot (D), is located on the baseline (C) and aligned to the follow-up OCT scan at 36 months (E). The presence of new subretinal fluid (SRF) is automatically identified by a SRF CNN and outlined in the annotated image at 36 months (purple area, F). This shows the cyan encircled yellow DARC spot (D) is predictive of new SRF 36 months later (E, F). Unique DARC spot prediction: Each individual DARC spot seen at baseline can be assessed in relation to the series of OCT scans performed at follow-up. Unique DARC spots associated with SRF (D) are encircled with different colours representing different time-points; including: 12-18 months (red, D, G), 18-24 months (light green, D, L), 24-30 months (purple, D, Q) and 30-36 months (yellow, D). These are shown with corresponding OCT scans at follow-up original (H, M, R) and baseline (J, O, T) with SRF CNN-defined newly formed SRF with the same colour-coding showing annotated new SRF areas (I, N, S) compared to baseline (K, P, U). The cyan line in each OCT scan highlights the vertical cross-section through the indicated DARC spot (white arrows; G, L, Q);

FIG. 2 shows that a CNN DARC Count>5 is associated with risk of developing SRF (wet AMD) in OCT over 36 months. (A) Plots showing the proportion of eyes remaining free of SRF (free of wet AMD) over 36 months, grouped by the CNN DARC count>5 (red) or ≤5 (blue). Eyes with a CNN DARC count>5 have a significantly increased level of developing SRF (Wilcoxon rank, p=0.0156) compared to those with a CNN DARC count≤5. (B) Table showing a breakdown of the conversion eyes by baseline diagnosis. All 7 eyes which went on to develop new SRF had CNN DARC counts>5, and 16 of the 17 eyes with either active choroidal neovascularisation (CNV) at baseline (n=10) or which converted on follow-up (n=7), had a CNN DARC count>5;

FIG. 3 shows CNN DARC counts significantly increased in eyes with large areas of SRF accumulation. Violin plots illustrating the distribution of data of unique DARC spots overlying SRF on OCT with the total area of accumulated SRF on OCT at each time point. To detect CNV lesions on the OCT, a CNN was used to identify areas of SRF, trained using data from The Retinal OCT Fluid Challenge (RETOUCH). To allow for the uneven number of OCT scans within 6-month intervals and between eyes, an average area of SRF was computed per interval. Eyes were divided into “high SRF” and “low SRF” using a threshold of 3000 um. A unique DARC count greater than 5 in all eyes, at all time points, is associated with large SRF accumulation. All p-values indicate level of Mann-Whitney's significance. Horizontal lines indicate medians and interquartile ranges with minimum and maximum points; and

FIG. 4 shows DARC identifies earliest changes of endothelial activity in a rabbit model of angiogenesis. A rabbit model of angiogenesis was created by intravitreal administration of 1 ug in 50 ul of human VEGF (hVEGF165—Peprotech, London UK) to the left eyes only of 3 rabbits. 2 and 4 days later intravenous ANX776 (0.2 mg) and 1% sodium fluorescein was given. Both eyes of rabbits were imaged using the ICGA and FFA settings of cSLO. DARC spots (identified as white spots which are ANX776 positive-labelled) are clearly visible in the left (A-C) hVEGF treated eyes compared to untreated right eyes (D-F). DARC counts were obtained by three masked manual observers. (G) Individual points represent separate manual observations of left and right eyes. All hVEGF eyes showed positive DARC staining compared to untreated, contralateral eyes. There was a significant difference (p=0.0203) between DARC counts in treated compared to control eyes. Inserts (A-F) show fluorescein angiography 4 days after treatment with hVEGF. All treated eyes went on to show fluorescein leakage at 4 days, (inserts A-C) compared to control (inserts D-F). Topical ranibizumab reduced angiogenic DARC labelling, (H-J). While there was a significant difference in DARC counts between treated and control eyes in A-F, this significance was abolished by ranibizumab treatment (N). Topical ranibizumab resulted in a significant reduction in DARC count, compared to vehicle controls (O, p=0.0262).

FIG. 5 shows that an anti-angiogenic treatment reduced vessel leakage in a rabbit model of angiogenesis. A rabbit model of angiogenesis was created as for FIG. 4. The anti-angiogenic treatment ranibizumab was topically applied. All control rabbit eyes treated with hVEGF had fluorescein leakage at 4 days (A-C), compared to ranibizumab eyes (G-I).

### EXAMPLES

### Background

Age Related macular degeneration (AMD) is the leading cause of blindness in developed countries with an incidence that is predicted to rise as the ageing population increases. Two forms of AMD exist, classified by the presence (“wet”) or absence (“dry”) of choroidal neovascularization (CNV) and characterized by formation of subretinal or intraretinal fluid (SRF or IRF, respectively) from leaking vasculature. Optical coherence tomography (OCT) is currently considered the gold standard for the management of AMD, especially when assessing therapy targeting anti-Vascular Endothelial Growth factor (anti-VEGF).

Detection of Apoptotic Retinal Cells (DARC) is a technique to monitor the rate of cell death in the retina that recently completed Phase I clinical trials for the diagnosis of Glaucoma (Cordeiro et al. 2017). The technology currently comprises intravenously administering a novel fluorescent agent called Anx776 which comprises a modified version of the endogenous protein Annexin A5, fluorescently conjugated to a near-infra red fluorophore Dy-776. Anx776 positive-labelled cells are visualised as “white spots” on the retina using the ICGA settings of a commonly utilised confocal scanning laser ophthalmoscope (cSLO). DARC utilised the unique optical properties of the eye to enable the possibility of directly observing single nerve cell apoptosis in patients using the fluorescent-labelled derivative of human Annex V. It was decided to investigate DARC as a prediction technique (i.e. before diagnosis can occur, due to an absence of sufficient symptoms) for the development of diseases, in particular wet-AMD.

### Materials and Methods

### Conjugation of D776 to Annexin 5

Recombinant bacterially expressed annexin 5 (5 mg in 1 ml isotonic buffer) was dialysed against ‘labelling buffer’ over three hours with two changes (250 ml each time). The labelling buffer was produced by dissolving 21 g (250 mmol) of sodium hydrogen carbonate in 400 ml distilled water. 1 g of sodium azide (0.2 percent) was added. The pH was adjusted with a concentrated aqueous solution of sodium hydroxide to pH 9.0. Prior to use, the buffer was diluted by addition of 9 parts water to one part of the concentrated stock solution (v/v). The annexin 5 protein is then ready for the labelling reaction. The dye to protein ratio was kept constant for each labelling reaction. Thus, 0.5 mg of the D776-NHS-ester was used for labelling 1 mg of annexin 5. This provides a molar ratio between dye and annexin 5 protein of approximately 500. The NHS-ester was dissolved in dimethylformamide by vortexing, and the reaction started by adding the dye to annexin 5 protein in the labelling buffer. The final concentration of annexin 5 in the labelling reaction was 5 mg/ml. Labelling was allowed to proceed in an Eppendorf tube placed in a shaker over the course of two hours.

At the end of the reaction period the coloured labelling solution was carefully pipetted onto a 5- or 10 ml column of Sephadex G-25 and allowed to seep into the gel. The conjugate was then eluted by slowly adding 100 mM PBS-buffer pH 7.4 drop by drop onto the column. The labelled protein runs ahead as a relatively sharp band while the free dye is slowly smearing behind. Once the conjugated annexin 5 arrives at the bottom of the column it was collected in an Eppendorf tube and is immediately ready for use.

### Clinical Trial Design

A single-centre, open-label, Phase 2 clinical trial of DARC was conducted at The Western Eye Hospital, Imperial College Healthcare NHS Trust. All subjects received a single intravenous injection of fluorescent annexin 5 (ANX776, 0.4 mg) between 15 Feb. 2017 and 30 Jun. 2017. AMD subjects, already under the care of the medical retina department at the Western Eye Hospital were recruited to the trial, with informed consent being obtained according to the Declaration of Helsinki after the study was approved by the Brent Research Ethics Committee. (ISRCTN10751859). All participants had DARC performed only once at baseline, with Optical Coherence Tomography (OCT) scans recorded at every follow up visit, as per standard of care, to a maximum of 36 months after DARC.

Patients were considered for inclusion in the study if they were eligible according to the inclusion and exclusion criteria outlined in Table 1. Specifically, for inclusion in the AMD cohort, they either had to show CNV (wet AMD) or changes consistent with “Early AMD” (drusen, retinal pigment epithelium (RPE) pigment changes) or “Late AMD” (geographic atrophy of the RPE (dry AMD)). Furthermore, the presence of ocular conditions with increased risk of choroidal neovascularisation (CNVM), and current or past use for more than 30 days of chloroquine, hydroxychloroquine, chlorpromazine, thioridazine, quinine sulfate, clofazimine, cisplatin, carmustine, (BCNU), deferoxamine, amiodarone, isoretinoin, or gold were excluded. Eyes were divided into those with active CNV, previous CNV, early AMD and late AMD at baseline, based on clinical examination and retinal imaging findings.

Of the 19 AMD subjects, 31 eyes were analysed with images at baseline and 240 minute time-points assessed.

### DARC Image Acquisition and Study Blinding

Retinal images were acquired with a cSLO (HRA+OCT Spectralis, Heidelberg Engineering GmbH, Heidelberg, Germany), as previously described in Normando et al. (2020). Fluorescence settings on the high-resolution ICGA mode were used (diode laser 786 nm excitation; photodetector with 800-nm barrier filter) with baseline infrared autofluorescent images were acquired after pupillary dilatation (1% tropicamide and 2.5% phenylephrine). All participants had DARC performed once only at baseline. Each human patient received ANX776 via intravenous injection (single dose 0.4 mg) and were imaged during and after ANX776 injection at 15, 120 and 240 minutes, with averaged images from 100 frames recorded at each time point. All images were anonymised before any analysis was performed, and only the 240 minute images analysed in this study.

A pilot study was also performed on 3 New Zealand White rabbits to assess DARC using an established model of retinal angiogenesis. All experiments were designed and conducted in accordance with the ARVO Statement for the Use of Animals in Ophthalmic and Vision Research and under protocols approved by the U.K. Home Office. Briefly, 2.5 kg rabbits were anesthetized with 50 mg/kg intramuscular injection of ketamine hydrochloride and 10 mg/kg xylazine, respectively. Pupils were dilated with 2.5% phenylephrine and 1% tropicamide. 50 ul containing 1 ug of humanVEGF165 (Sigma, UK) dissolved in 0.1% bovine serum albumin (Sigma UK) was injected intravitreally into the left eye only of each animal. 48 hours later, under general anaesthesia, rabbits received intravenous Anx776 (0.2 mg) and 1% sodium fluorescein and underwent DARC and 448 imaging at 40 minutes using high-resolution ICGA and FFA modes of the HRA-Spectralis. 3 masked observers performed manual counts on DARC images. For anti-angiogenic experiments, 3 rabbits were then administered ranibizumab eye drops. 3 rabbits were vehicle controls.

### OCT Scan Acquisition

OCT scans were recorded at every follow up visit of each human patient, as per standard of care, to a maximum of 36 months after DARC. DARC images and OCT scans were assessed in a post-hoc analysis.

### DARC Spot Detection

The full method for spot detection is described in Normando et al. (2020). Briefly, this comprised training a central neural network (CNN) to classify spots from a control dataset which were initially identified using template matching and manually classified by 5 observers. Agreement of 2 out of 5 observers was used to train the CNN, which was found to have an accuracy of 97%, with 91.1% sensitivity and 97.1% specificity.

The CNN model and weights that were reported have been used in this work with no modifications.

### Alignment of CLSO and OCT

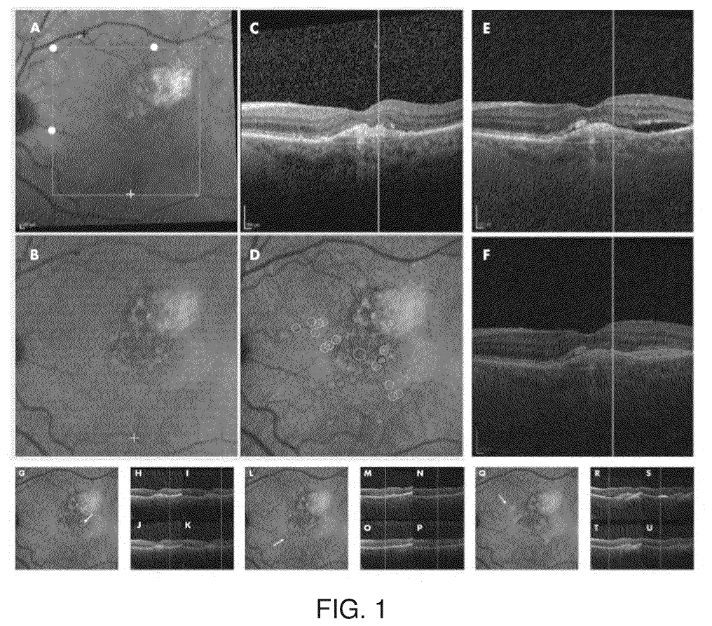

DARC images of each eye were aligned with the corresponding OCT series of scans, allowing for the location of a DARC spot to be identified on the corresponding OCT scan at subsequent follow up. This alignment also allowed for the location of the identified DARC spots in the CLSO images to be referenced against the location of sub-retinal fluid (SRF) within the OCT images. To register the DARC CLSO images with OCT images, briefly, baseline autofluorescence and DARC images (240 min) were aligned automatically using an affine transformation followed by a non-rigid B-spline transformation. This was completed using multi resolution alignment methods within SimpleITK (NumFOCUS, US) and a gradient descent optimiser. The reflectance image of sequential Spectralis OCT scans were next aligned with the registered DARC image, by manual placement of fiducial markers (white spots, FIG. 1) on the CLSO autofluorescence and OCT reflectance images, then calculating and applying an affine transform. Once all the images were aligned then a location on the 240 minute DARC image could be selected with the same location being identified in corresponding OCT image slices.

### CNN to Identify SRF

To detect choroidal neovascularization (CNV) lesions on the OCT, a CNN was used to identify areas of SRF. The CNN was trained using data from The Retinal OCT Fluid Challenge (RETOUCH), described in Bogunovi et al. (2019).

A custom UNET as described by Ronneberger et al. 2015 was adapted. A general training mechanism for a traditional UNET has been previously implemented and this was trained using data from The Retinal OCT Fluid Challenge (RETOUCH) described in Bogunovi et al. (2019), where Intraretinal fluid (IRF), Subretinal fluid (SRF), Pigment Epithelial Detachment (PED) area have been manually annotated by multiple observers.

A 3-channel label mask for each OCT scan image was created such that each annotated type was represented in a separate channel of a three channel RGB image. The model was trained on a Windows PC with an 8 Gb NVIDIA GTX 1080 graphics card and was trained for 100 epochs using the UNET with a fixed image size of 512×512, 32 filters and a dropout of 0.2 and only horizontal flipping was randomly augmented. The training data set was separated approximately ⅔ (N=4,624) for training and ⅓ (N=2,312) for validation, the image size of each of the SRF scan images was 497×495 pixels.

Following the completion of model training the model was tested on ⅓ validation set. Comparing the DICE coefficient (as described in Normando et al. 2020) obtained from the trained network, for SRF this compared favourably with the best reported system reported in RETOUCH which gave a DICE coefficient score of 0.83 for Spectralis vs the reported 0.75. Although a direct comparison could not be made as the reported results were on the challenge dataset on combined Cirrus, Spectralis and Topcon vs our results on a partitions subset of the training data set.

To ensure that results obtained using the RETOUCH dataset were representative of our own DARC study, 735 anonymised OCT images were manually annotated by an senior retinal specialist using VGG Image Annotator (VIA), Visual Geometry Group, Oxford University and https://gitlab.com/vgg/via/blob/master/Contributors.md. The DICE coefficient was calculated from using the SRF CNN described to be 0.84. The SRF CNN was then applied to the 20,630 OCT image from the dataset of 31 eyes.

A DARC spot was said to intersect with an area of SRF is there was any SRF identified normal the OCT scan plane.

### Calculation of SRF Accumulation

The presence of SRF at 6-monthly intervals was used to classify eyes. To allow for the uneven number of OCT scans within 6-month intervals and between eyes, an average area of SRF was computed per interval. New SRF was defined as eyes with SRF on OCT that were classified as SRF-negative in the preceding time period.

For any 6 month time-interval, SRF amount for each eye was taken to be the normalised sum of the detected SRF pixels for all the oct scans for a specific time point for the whole eye. The accumulated SRF was the volume of SRF per OCT scan, averaged by the number of OCT scans in that 6-month period.

### Statistical Analysis

Statistical analysis was performed using GraphPad Prism (version 8.01), Sklearn (version 0.0), SPSS (IBM SPSS version 25) and Python (version 3.6). Precision-recall curves were constructed with precision, recall, F1 score and area under the curve (AUC), generated for SRF CNN testing. Agreement between clinical and CNN definitions of SRF were assessed with kappa statistics. Graphs were constructed based on eyes remaining free of SRF comparing eyes with a total CNN DARC count above and below 5, and compared using Wilcoxon rank pairing for each time point and simple linear regression statistics. Confusion matrices were constructed to assess prediction rate of eyes with new SRF using only DARC spots overlying SRF, from which a positive predictive value (PPV), specificity, sensitivity were calculated. Correlation of unique DARC spots overlying SRF on OCT with the total area of accumulated SRF on OCT at each time point was made using Spearman's correlation coefficient (p<0.05). Assessment of DARC spots overlying SRF at each OCT time point was performed using violin plots, dividing groups into low and high areas of accumulated SRF. SRF high and low groups were compared using Mann Whitney tests. Manual DARC counts were analysed using paired t-test comparing treated human-VEGF left to untreated right eyes of rabbits.

### Example 1: DARC Predicts New Subretinal Fluid (Wet AMD) in OCT at 36 Months

We decided to investigate DARC as a prediction technique (i.e. before diagnosis can occur, due to an absence of sufficient symptoms) for the development of diseases, in particular wet-AMD.

A post-hoc analysis of DARC results from a cohort of 19 AMD patients in the Phase 2 DARC clinical trial at the Western Eye Hospital, ICNHT, London was performed. Patients were followed up regularly with OCT scans for up to 36 months after DARC was performed. Patient demographics are shown in Tables 2 and 3. Of the original 31 eyes assessed with DARC, OCT follow-up was available for 29 eyes followed up regularly with OCT scans for up to 36 months after DARC was performed. The mean number of OCT scans per eye per 6 month interval ranged from 1.70 at 30-36 months to 2.59 at 12-18 months. As patients continued treatment according to standard of care, the mean number of anti-VEGF intravitreal injections received varied from 2.0 at 30-36 months to 3.61 at 0-6 months.

Analysis at each time point was performed to see if a DARC spot from baseline predicted the occurrence of wet AMD by its intersection with any area of new SRF on OCT as shown in FIG. 1, as detected by a CNN. Using the SRF CNN to define new SRF, eyes were divided into new wet AMD and no wet AMD groups at each timepoint.

The occurrence of new SRF at each time period is shown in Table 4, with conversion rates of 7.4, 11.5, 26.1, 26.1, 29.2 and 33.3% at 6, 12, 18, 24, 30 and 36 months respectively, using only the definition of OCT CNN SRF. Eyes were classified as “converted” only if they showed new SRF for the first time. The CNN DARC count was greater than 5 in all but one eye associated with SRF formation.

Both auto-fluorescent and DARC spots overlying SRF in the baseline OCT were excluded from a further analysis, where only the first occurrence of individual DARC spots overlying new SRF (termed “unique DARC spot”) were included to avoid repeat counting. At a specific time-point, if an eye showed any unique DARC spots overlying new SRF on OCT, it was classified as true positive; an eye with SRF but no unique DARC spots was false negative; an eye with no SRF but with unique DARC spots was false positive; and eyes with no SRF or DARC spots were true negative. Confusion matrices (with each confusion matrix determining the prediction decision of DARC CNN by eye at each time interval) were constructed for each 6 monthly follow-up period from 6 to 36 months after baseline. Table 4 summarizes the results of the confusion matrices, showing the ability of unique DARC spots to predict eyes with new SRF activity. Hence, at 6 months, the system had a specificity of 90%, a sensitivity of 83%, a PPV of 71% and an NPV of 95%. The PPV of the DARC DL system is over 70% at all time points, reaching a peak of 86% at 30 months. The specificity ranges from 79 to 90% with sensitivities above 80%.

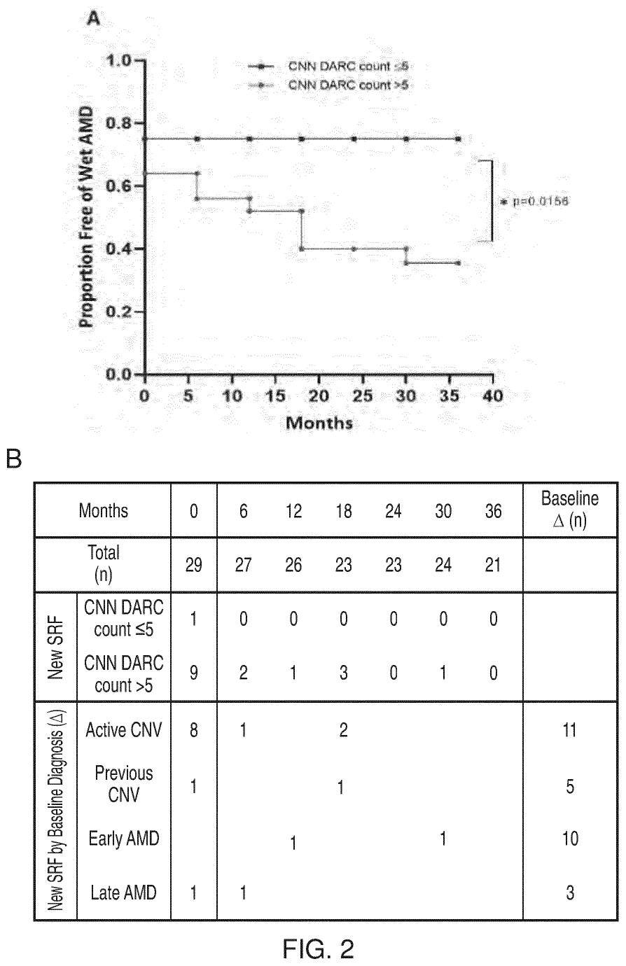

FIG. 2A shows the proportion of eyes remaining free of SRF (free of wet AMD) at each 6 month time period, and grouped by the CNN DARC count threshold of 5. Comparison between the groups showed that eyes with a CNN DARC count>5 showed a significantly increased level of developing SRF (Wilcoxon rank, p=0.0156) compared to those with a CNN DARC count≤5. Linear regressions of the same data was also performed and the slopes of the regression estimates were highly statistically significantly different (p<0.0001, R²=0.92 DARC CNN>5 compared to R²=1.0 DARC count≤5), indicating a “time*group” effect, that is, that the progress of time affects the CNN DARC count>5 group significantly differently than it does the CNN DARC count≤5 group. Observation of the fit line and the data points suggests that the greater hazards occur in the first 16 months, with hazard being relatively flat after that whereas a CNN DARC count≤5 was significantly associated with increased SRF-free survival.

A breakdown of the conversion eyes by baseline diagnosis is shown in FIG. 2B. All 7 eyes which went on to develop new SRF had CNN DARC counts>5. 16 of the 17 eyes with either active CNV at baseline (n=10) or which converted on follow-up (n=7), had a CNN DARC count>5.

This shows that DARC can be used to predict the development of wet AMD, thereby ensuring early diagnosis.

### Example 2: DARC has Increased Accuracy of Prediction Relative to Other Prediction Methods

We then compared the results from our DARC+OCT model of Example 1 to that reported by Yim et al. (2020). Yim described the use of deep learning (DL) with OCT to improve predicting the development of choroidal neovascularisation (CNV) or wet AMD at 6 months by analysing OCT features. However, low predictive values were reported, namely, a specificity of 55% at 80% sensitivity, a sensitivity of 34% at 90% specificity, and a positive predictive value (PPV) of 7.3% and 13.2%, respectively.

Table 5 shows a comparison of results from our DARC DL model of Example 1 to that reported by Yim et al. (2020). The PPV of the DARC DL system is much greater, being 100% at 6 months compared to the Yim system of 7% or 13%. This is also seen with the consistently greater specificity and sensitivity readings of DARC DL, being 100% and 50% at 6 months, and 95% and 83% at 36 months, respectively. The conversion rates in Example 1 are similar to that of Yim et al. at 6 months, these being 14.8% and 13.5% respectively. This is despite a difference in sample size.

### Example 3: An Increased DARC Count Correlates to the Magnitude of SRF Accumulation Observed

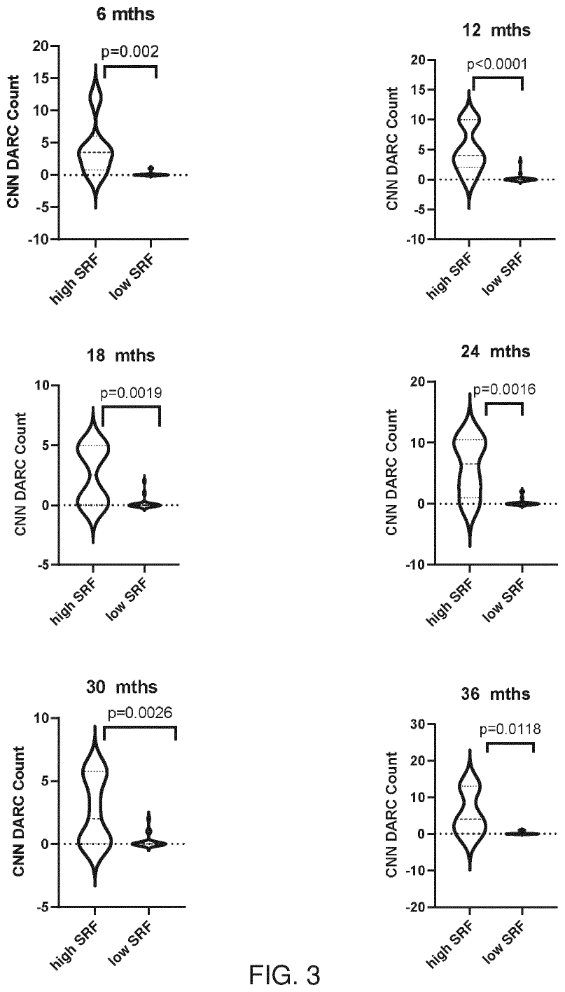

We hypothesised that the DARC DL system performed better than the Yim system, due to its ability to assess single cell activity. To investigate this further, the relationship of the DARC count with the magnitude of SRF accumulation for each eye at each time-point was evaluated. Eyes were divided into “high SRF” and “low SRF” using a threshold of 2000 pixels at each time point. FIG. 3 shows that DARC counts overlying SRF are significantly (p=0.002, <0.0001, 0.0019, 0.0016, 0.0026 and 0.0118 at 6, 12, 18, 24, 30 and 36 months respectively) increased in eyes with large areas of SRF accumulation at each time points. Hence, the greater the SRF, the higher the DARC count, with all eyes having a unique DARC count greater than 5 associated with large SRF accumulation. Indeed, the DARC spots overlying SRF at each time point was positively correlated with the OCT SRF area at 6 (Spearman's r=0.65, p=0.0001), 12 (r=0.72, p<0.0001), 18 (r=0.60, p=0.0006), 24 (r=0.57, p=0.0012), 30 (r=0.67, p<0.0001) and 36 (r=0.59, p=0.0008) months. This is supported by single DARC spots predicting new areas of SRF, as illustrated in FIG. 1.

This confirms that the greater the DARC count, the greater the severity of wet-AMD which develops.

### Example 4: DARC Identifies Early Stage Angiogenesis in Rabbits

A rabbit model of angiogenesis was created by intravitreal administration of 1 ug in 50 ul of human VEGF (hVEGF165-Peprotech, London UK). 48 hours later intravenous ANX776 (0.2 mg) was given and rabbits were imaged using the ICGA settings of cSLO.

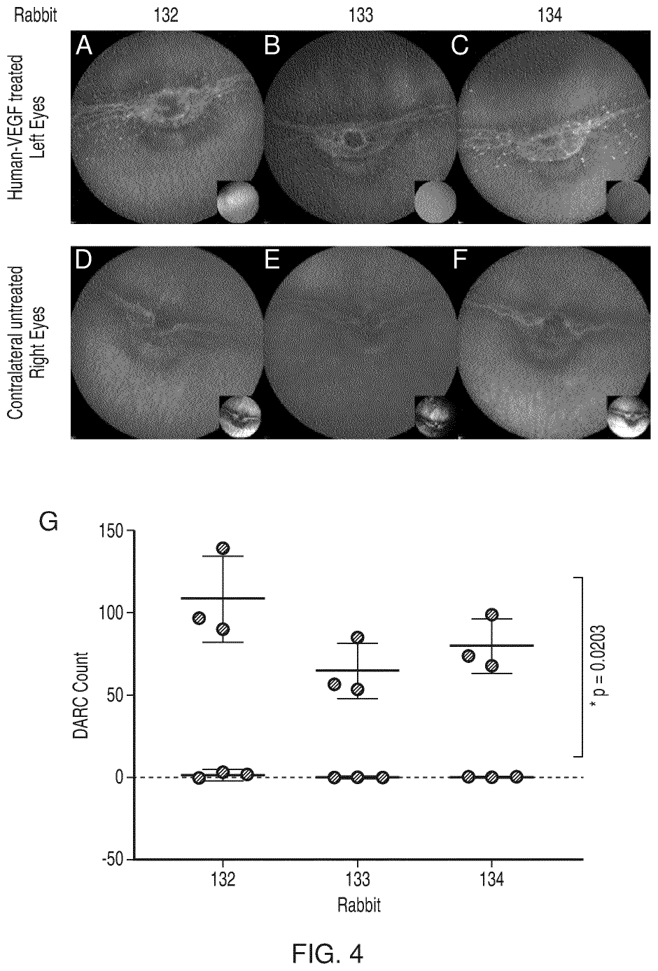

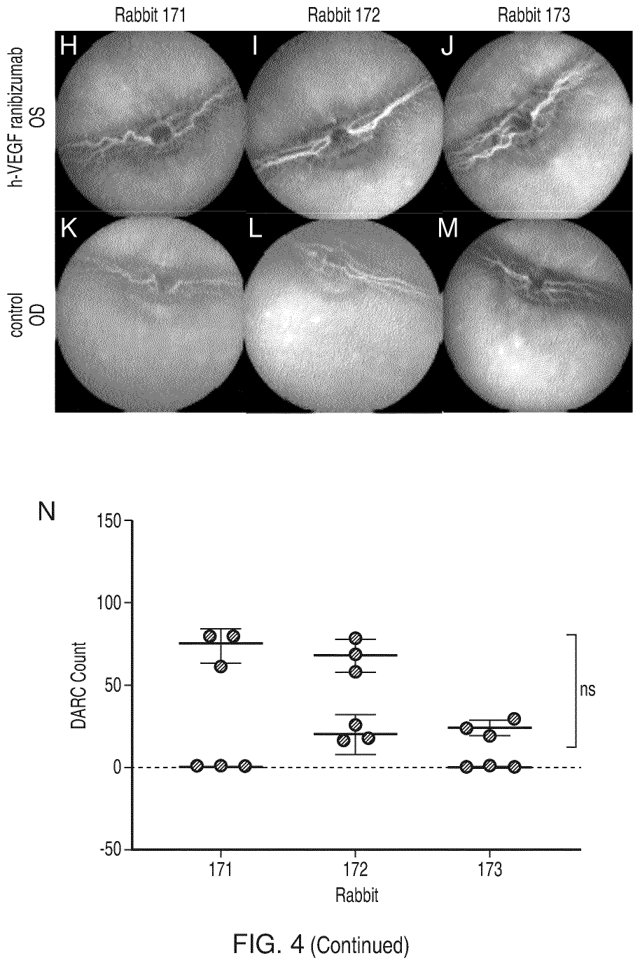

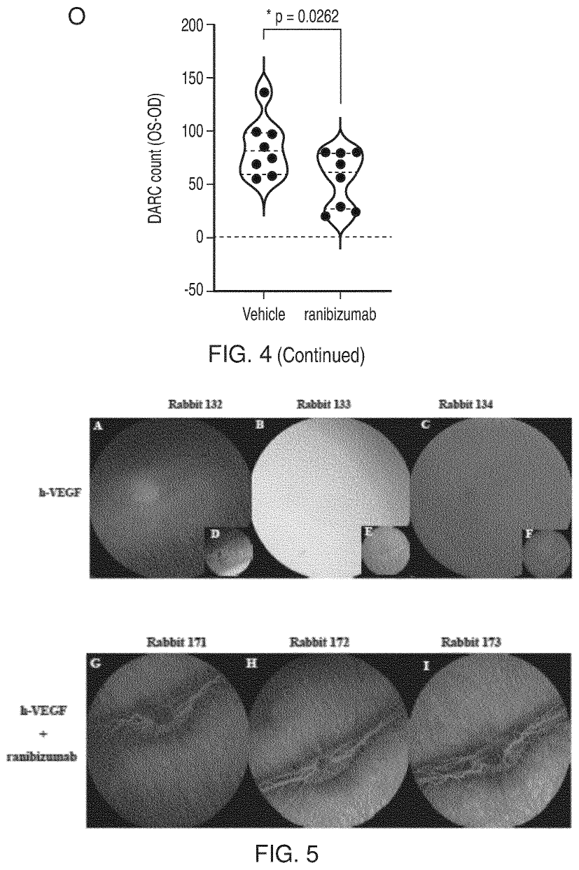

All rabbit eyes treated with hVEGF were found to have DARC positive-staining compared to the untreated contralateral eye 2 days after treatment. FIG. 4 shows DARC images taken of both eyes of each animal, with DARC spots clearly visible in the left (A-C) hVEGF treated eyes compared to untreated right eyes (D-F), with a significant increase (p=0.0203, G) in treated eyes. All treated eyes went on to show fluorescein leakage at 4 days, as shown in the inserts A-C compared to control (inserts D-F).

This further confirms the predictive and identification value of DARC for early stage angiogenesis.

### Example 5: DARC can be Used to Monitor the Efficacy of Anti-Angiogenic Treatment

Rabbit models of angiogenesis, as described in Example 4, were topically treated with the anti-angiogenic treatment ranibizumab. Angiogenic DARC labelling was reduced by topical ranibizumab (FIG. 4H-J). Topical ranibizumab resulted in a significant reduction in DARC count, compared to vehicle controls (FIG. 40, p=0.0262).

Confirmation of the efficacy of topical treatment with ranibizumab is shown in FIG. 5. All control rabbit eyes treated with hVEGF had fluorescein leakage at 4 days (FIG. 5A-C) compared to ranibizumab eyes (G-I). Images were collected 8 minutes (D-F) and 20 minutes (A-C, G-I) after 1% sodium fluorescein administration. This figure confirms that topical ranibizumab reduced vessel leakage in vivo. This, in turn, confirms the ability of DARC to monitor the efficacy of treatment.

TABLE 1
Inclusion and exclusion criteria
INCLUSION CRITERIA

| | |
|---|---|
| 1. | Age ≥18 years. |
| 2. | Clear optical media in the studied eye. |
| 3. | Refractive error not higher than spherical equivalent of 10 D and |
| | best corrected visual acuity equal to 6/24 or better at qualification. |
| 4. | Women of childbearing potential identified as not |
| | pregnant and have consented to complete a pregnancy test. |
| 5. | Subjects who have capacity to consent, have personally signed |
| | and dated the informed consent document indicating that they |
| | have been informed of all pertinent aspects of the study. |

EXCLUSION CRITERIA

| | |
|---|---|
| 1. | Presence of severe, unstable or uncontrolled systemic disease. |
| 2. | Known intolerance to IMP. |
| 3. | Body weight <40 kg or >150 kg. |
| 4. | Inability to comply with the study or follow-up procedures. |
| 5. | Any subjects with a known history of clotting diseases |
| | (including DVTs), and subjects taking anticoagulants. |
| 6. | Ocular surgery within the past 3 months or planned surgery |
| | in the study eye, during the course of the trial. |
| 7. | Pregnant or lactating, or not using adequate contraception* |
| | for the duration of the trial (and 30 days post injection of |
| | study drug). |
| 8. | Currently being treated for cancer or any other disease |
| | likely to adversely affect participation in this study. |
| 9. | AIDS/HIV. |
| 10. | History of alcoholism or drug addiction. |
| 11. | History or active uveitis. |
| 12. | History of systemic vasculitis, collagenosis or ongoing |
| | treatment of cancer. |
| 13. | Evidence of previous retinal vascular disease. |
| 14. | Individuals with terminal illness, or mental illness |
| | affecting their compliance with the study |
| 15. | Any other disease, condition or laboratory abnormality |
| | that in the opinion of the CI may increase the risk for the |
| | participation or may interfere with the interpretation of |
| | study results and in the judgement of the Investigator would |
| | make the subject inappropriate for entry into the study. |
| 16. | Central corneal thickness <450 pm or >650 pm. |
| 17. | Currently, or within the last 3 months, enrolled in a |
| | clinical trial of an investigational medicinal product. |
| 18. | History of retinal laser photocoagulation. |
| 19. | Media opacities or retinal pathology or amblyopia significantly |
| | limiting visual acuity, visual field test or retinal imaging. |
| 20. | Any other condition or pathological process that in the opinion of |
| | the investigator would not make the patient suitable for the trial. |

TABLE 2
Demographics and baseline characteristics

| Baseline Characteristic | | | Patients/Eye | |
|---|---|---|---|---|
| Age | Mean years (SD) | | 82.89 | (5.84) |
| Gender | Males no (%) | | 11 | (57.89) |
| | Females no (%) | | 8 | (42.11) |
| | Total | | 19 | |
| Ethnicity | Caucasian (%) | | 15 | (78.95) |
| | Black (%) | | 0 | |
| | Hispanic (%) | | 0 | |
| | Asian (%) | | 4 | (21.05) |
| | Other (%) | | 0 | |
| Vital | Mean Blood | Systolic, | 142 | (17.5) |
| signs | Pressure | mmHg (SD) | | |
| | | Diastolic, | 71 | (9.6) |
| | | mmHg (SD) | | |
| | Heart Rate | Beats/min (SD) | 65 | (9.7) |
| | Respiratory Rate | Resp/min (SD) | 17 | (1.4) |
| | Height | Metres (SD) | 1.62 | (0.1) |
| | Weight | Kg (SD) | 65.8 | (9.9) |
| Study Eye | Right (%) | | 3 | (15.79) |
| | Left (%) | | 4 | (21.05) |
| | Both (%) | | 12 | (63.16) |
| | Total | | 31 | (100) |
| BCVA | Affected eye | Logmar Mean (SD) | 0.39 | (0.41) |
| | Unaffected eye | | 0.27 | (0.4) |
| IOP | Affected eye | mmHg (SD) | 13.5 | (3.55) |
| | Unaffected eye | | 13.7 | (2.88) |

TABLE 3
Follow-up summary

| | | |
|---|---|---|
| Intervention | Mean | (SD) |

| | | |
|---|---|---|
| Study Eye | Total | 19 |

| | | | | |
|---|---|---|---|---|
| OCT scans | 0-6 | months | 2.48 | (1.67) |
| | 6-12 | months | 2.45 | (1.86) |
| | 12-18 | months | 2.59 | (1.52) |
| | 18-24 | months | 2.37 | (1.36) |
| | 24-30 | months | 2.15 | (1.38) |
| | 30-36 | months | 1.70 | (1.32) |

| | | | |
|---|---|---|---|
| Anti-VEGF Injections | Before DARC | 15.58 | (13.90) |

| | | | |
|---|---|---|---|
| 0-6 | months | 3.61 | (0.60) |
| 6-12 | months | 2.94 | (1.00) |
| 12-18 | months | 2.33 | (1.40) |
| 18-24 | months | 2.39 | (0.77) |
| 24-30 | months | 2.25 | (1.29) |
| 30-36 | months | 2.00 | (0.94) |

TABLE 4
Accuracy of unique DARC spots predicting eyes with new SRF

| Predictive | Time (months) | | | | | |
|---|---|---|---|---|---|---|
| Value % | 6 | 12 | 18 | 24 | 30 | 36 |
| | DARC | DARC | DARC | DARC | DARC | DARC |
| PPV | 71 | 70 | 82 | 77 | 86 | 80 |
| Specificity | 90 | 86 | 89 | 82 | 86 | 79 |
| Sensitivity | 83 | 88 | 82 | 83 | 80 | 80 |
| NPV | 95 | 95 | 89 | 88 | 80 | 79 |
| % Conversion | 7.4 | 11.5 | 26.1 | 26.1 | 29.2 | 33.3 |
| Rate | | | | | | |

TABLE 5
Comparison of predictive values of DARC DL and Yim DL systems and published conversion rates for developing wet AMD

| | Time (months) | | | | | | | | | | | |
|---|---|---|---|---|---|---|---|---|---|---|---|---|
| | 6 | | | 12 | | | 18 | 24 | | | 30 | 36 |
| % | DARC DL | YIM DL | | DARC DL | YIM DL | | DARC DL | DARC DL | YIM DL | | DARC DL | DARC DL |
| | SYSTEM | SYSTEM | | SYSTEM | SYSTEM | | SYSTEM | SYSTEM | SYSTEM | | SYSTEM | SYSTEM |
| Sensitivity | 50 | 80 | 34 | 70 | 80 | 29 | 80 | 85 | 80 | 30 | 85 | 95 |
| Specificity | 100 | 55 | 90 | 100 | 54 | 90 | 89 | 80 | 52 | 90 | 66 | 83 |
| PPV | 100 | 7 | 13 | 100 | 16 | 23 | 94 | 89 | 33 | 47 | 85 | 95 |
| NPV | 52 | 98 | 97 | 53 | 10 | 92 | 57 | 67 | 90 | 81 | 55 | 45 |

| | DARC DL | Yim¹ | DARC DL | Fasler ⁴ | DARC DL | DARC DL | Misc ³⁻⁵ | DARC DL | DARC DL |
|---|---|---|---|---|---|---|---|---|---|
| Conversion Rate % | 14.8 | 13.5 | 23.1 | 21.0 | 25.0 | 17.4 | 16.6 - 32.0 | 25.0 | 38.1 |

### REFERENCES

Bogunovi, H. et al. RETOUCH—The Retinal OCT Fluid Detection and Segmentation Benchmark and Challenge. XX, (2019).
Cordeiro, M. F. et al. Real-time imaging of single neuronal cell apoptosis in patients with glaucoma. Brain, 274, 61 (2017).
Normando, E. M. et al. A CNN-aided method to predict glaucoma progression using DARC (Detection of Apoptosing Retinal Cells). Expert Rev. Mol. Diagn. 00, 1-12 (2020).
Lindekleiv, H, Erke MG. Projected prevalence of age-related macular degeneration in Scandinavia 2021-2040. Acta Ophthalmol. 91 (4): 307-311 (2013).
Ronneberger, O., Fischer, P. & Brox, T. U-Net: Convolutional Networks for Biomedical Image Segmentation. 1-8 (2015).
Saint-Hubert et al. Molecular imaging of cell death. Methods. 48 (2), 178-87 (2009).
Tait et al. Structural requirements for in vivo detection of cell death with 99mTc-annexin V. J Nucl Med. 46 (5), 807-815 (2005).
Yim, J. et al. Predicting conversion to wet age-related macular degeneration using deep learning. Nat. Med (2020). Doi:https://doi.org/10.1038/s41591-020-0867-7

1-14. (canceled)

15. A method of identifying or predicting angiogenesis in a subject, the subject having been administered a labelled cell stress marker, comprising the steps of:
(a) generating an image from the subject using a first imaging device;
(b) identifying labelled cell stress marker positive cell(s) in the image;
wherein angiogenesis is identified or predicted if labelled cell stress marker positive cell(s) are identified in the image.

16. The method of claim 15, wherein the cell stress marker is an annexin or a fragment or variant thereof.

17. The method of claim 16, wherein the cell stress marker is annexin 5, 11, 2 or 6.

18. The method of claim 15, wherein the label is fluorescent.

19. The method of claim 18, wherein the fluorescent label has a wavelength in the infra-red spectrum.

20. The method of claim 18, wherein the fluorescent label is D-776.

21. The method of claim 15, wherein the angiogenesis is physiological, therapeutic or pathological angiogenesis.

22. The method of claim 21, wherein the physiological angiogenesis is associated with previous exercise by the subject.

23. The method of claim 21, wherein the therapeutic angiogenesis is associated with wound healing and/or integration of a tissue engineered scaffold.

24. The method of claim 20, wherein the pathological angiogenesis is associated with a disease, a stage of disease, is predictive of disease developing or of disease severity.

25. The method of claim 24, wherein the pathological angiogenesis is predictive of disease developing.

26. The method of claim 24, wherein the disease comprises cancer, autoimmune disease, cardiovascular disease, allergic disease, sickle cell disease, and/or ocular disease.

27. The method of claim 26, wherein the disease comprises ocular disease.

28. The method of claim 27, wherein the ocular disease comprises age-related macular degeneration (AMD), optionally wet-AMD.

29. The method of claim 24, wherein the method further comprises administering an appropriate treatment for the disease, stage of disease, disease severity or predicted disease identified.

30. (canceled)

31. The method of claim 15, wherein identifying labelled cell stress marker positive cell(s) comprises counting the number of labelled cell stress marker positive cell(s) in the image.

32. The method of claim 31, wherein the identification of labelled cell stress marker positive cell(s) is computer implemented.

33. The method of claim 31, wherein the method further comprises determining a location of the labelled cell stress marker positive cell(s) in the subject.

34. The method of claim 33, wherein the location is determined using another image obtained using a second imaging device.

35. The method of claim 34, wherein the second imaging device comprises a computerized tomography (CT) scanner, a magnetic resonance imaging (MRI) scanner, a position emission tomography (PET) scanner, a polariser, a reflective image scanner, fundus camera and/or an OCT scanner.

36. The method of claim 35, wherein the second imaging device comprises the OCT scanner.

37. The method of claim 15, wherein the first imaging device comprises a confocal scanner laser microscope, optionally a confocal scanning laser ophthalmoscope (cSLO).

38-39. (canceled)
